(19) United States
(12) Patent Application Publication
(10) Pub. No.: US 2005/0101964 A1
(43) Pub. Date: May 12, 2005

(54) Spinal fusion procedure using an injectable bone substitute

(75) Inventors: Ruey-Mo Lin, Tainan (TW); Jiin-Huey Chern Lin, Winnetka, IL (US); Chien-Ping Ju, Carbondale, IL (US)

Correspondence Address: MEYERTONS, HOOD, KIVLIN, KOWERT & GOETZEL, P.C., P.O. BOX 398, AUSTIN, TX, 78767-0398, US

(73) Assignee: Calcitec, Inc., Austin, TX (US)

(21) Appl. No.: 10/982,660

(22) Filed: Nov. 4, 2004

Related U.S. Application Data
(60) Provisional application No. 60/518,475, filed on Nov. 7, 2003.

(51) Int. Cl. A61F 2/38; A61F 2/00

(52) U.S. Cl. 606/92

(57) ABSTRACT

Methods for performing spinal fusions using an injectable calcium phosphate-based bone substitute are provided. The injectable bone substitute is injected into the anterior portion of an interbody space and allowed to solidify in vivo. The injectable bone substitute has a minimum compression strength of 10 MPa after setting for about 30 minutes and preferably solidifies to a compression strength of 25 MPa within 24 hours of injection. Optionally, the posterior portion of the interbody space is fixed using a metallic implant selected from rods and pedicle screws or plates and pedicle screws by attachment thereof to adjacent vertebrae.

### CROSS REFERENCE TO RELATED APPLICATIONS

This application claims the benefit of priority under 35 U.S.C. § 119(e) of U.S. Application No. 60/518,475, filed Nov. 7, 2003, the content of which is incorporated herein by reference.

### FIELD OF THE INVENTION

The invention relates generally to methods of performing spinal fusion surgery and more specifically to methods of performing spinal fusion surgery using a bone substitute.

### BACKGROUND INFORMATION

A variety of interbody implants are available for spinal fusion procedures. These implants have been manufactured of various materials including steel, titanium, composites, allograft, xenograft or other biocompatible materials, and have the necessary strength to prevent the interbody space from collapsing before fusion has occurred. Other techniques for spinal fusion include the placement of bone graft material in the interbody space along with a plate or rod construct that spans the affected interbody space. Once fusion has occurred, the implants and hardware used to maintain the stability of the segment remain in the body to aid in stabilizing the spine.

Other types of implants have been developed from bio-compatible metals which incorporate threads on the outer surface of the implant that retain the implant in the interbody space after it is threaded therein. Still other implants have been developed that are made from bone. Examples of such spacers made from bone having use in spinal procedures are disclosed in U.S. Pat. No. 5,989,289. These spacers are provided with vertebral engaging surfaces on the upper and lower faces of the implant to resist migration of the implant in the interbody space and/or expulsion of the implant from the interbody space. While spacers made of bone can be readily incorporated in fusion procedures, the inherent brittle nature of bone resulting from a high mineral content, particularly load-bearing cortical bone, may limit its potential for use in applications that require the implant to resist loading. For example, cortical bone typically consists of approximately 70% mineral content and 30% non-mineral matter. Of this non-mineral matter, approximately 95% is type I collagen, with the balance being cellular matter and non-collagenous proteins.

The procedure used for fusing both the posterior and anterior elements of an unstable spinal location simultaneously is known as a 360° fusion. The most common method used for 360° fusion, following preparation of the interbody space, as needed, is to use metallic implants such as rods and screws, or plates and screws to fix the posterior elements of the interbody space. The anterior elements are typically fused using either solid allograft/autograft bone dowels/plugs (cortical and cancellous components), or a metallic/carbon “cage” implant filled with autograft, allograft or a bone substitute material.

Bone grafts have commonly been used in a fixed shape, pulverized, or as pliable demineralized bone. One form of a pliable bone graft is a demineralized bone material typically in the form of a sponge or putty having very little structural integrity. While a demineralized bone segment may retain properties suitable to support bone ingrowth, the structural properties of the bone are altered by removal of its mineral content. Thus, such bone sponges and putties may not typically be used in load-bearing applications without assistance from a plate or rod construct that spans the affected interbody space.

Therefore, there remains a need for new methods for performing spinal fusion that result in implants having the requisite load carrying capabilities.

### SUMMARY OF THE INVENTION

The invention is based on the discovery that an injectable calcium phosphate-based bone substitute can be used to perform single, or multi level spinal fusions, such as 360° spinal fusions used in treatment of degenerative disc disease.

In one embodiment, the invention provides methods for performing one or more spinal fusions in a subject comprising introducing an effective amount of an injectable calcium phosphate-based bone substitute into one or more interbody spaces in the subject by injection through a syringe, catheter, or cannula to facilitate single, or multi level spinal fusion.

In another embodiment, the invention provides method for performing one or more spinal fusions on a subject by placing in the posterior portion of at least one suitable interbody space a metallic implant selected from rods and pedicle screws or plates and pedicle screws by attachment to adjacent vertebrae. An effective amount of a calcium phosphate-based bone substitute is injected into the anterior portion of the interbody; and allowed to solidify in vivo, thereby performing the spinal fusion.

### DETAILED DESCRIPTION OF THE INVENTION

The present invention is based on the discovery that an injectable calcium phosphate-based bone substitute can be used to facilitate bone fusion, such as 360° spinal fusion.

Patient Preparation

The subject is placed in the prone position on the operating table. The subject is then prepped and draped to allow surgical access to diseased spine level(s). General anesthesia is administered. Surgical approach to targeted spine motion segment level(s) and incision site are confirmed by x-ray.

Surgical Approach

Using a posterior midline incision over the diseased motion segment(s), tissue is dissected to expose the spinous process and lamina of the targeted posterior spine level(s). Any pathology causing symptomatic claudication of the posterior spine neural anatomy is removed. Removal of suspect tissue may require a laminectomy, laminotomy or foramenotomy and resection/dissection of the ligamentum flavum. It is possible that neural compression is transient, resulting from an unstable motion segment. Instability can be caused by the collapse of the interbody space due to disc degeneration. In this case, it may not be necessary to decompress tissue, but only to stabilize the motion segment(s).

Once the decompression is complete, the motion segment may need to be mechanically stabilized. Instability is checked by manipulation of the spine. Even if instability exists with no decompression, the spine will still need to be stabilized.

Disc degeneration is now commonly treated with a 360° motion segment fusion, whereby both the anterior and posterior spine elements of the interbody space are fused. It is important to consider the mechanical stresses place on the anterior and posterior elements when considering a fusion technique. The anterior motion segment elements (vertebral bodies and disc) bear approximately 80% of the compressive force at that given level in the spine. The posterior ⅓ of the vertebral body and disc represent the center point for axial compression in the spine. These mechanics are critical for assessing what type of fusion will have the best clinical outcome for a given pathology. In cases where a 360° fusion procedure is deemed the best technique, the invention provides a spinal fusion procedure in which an injectable bone substitute with a suitable compressive strength profile is used for the anterior portion of the fusion.

360° Fusion Technique

In one embodiment, the invention methods for performing one or more spinal fusions in a subject include introducing an effective amount of an injectable calcium phosphate-based bone substitute into one or more suitable interbody spaces in the subject by injection through a syringe, catheter, or cannula to facilitate single, or multi level spinal fusion. The bone substitute is allowed to set under physiological conditions, i.e., in vivo, over time. Preferably, the bone substitute sets by hardening to form a solid mass and allows ingrowth of autologous bone in vivo over time. The methods for fixation of the interbody space in its posterior portion can be any method known in the art. The new aspects of the invention methods reside in the technique applied to the anterior portion of the fusion as well as in the combination of known standard posterior fixation techniques with the new methods for using an injectable calcium phosphate-based bone substitute to fill the anterior interbody space.

In another embodiment, the invention methods for performing one or more spinal fusion on a subject comprise placing in the posterior portion of at least one suitable interbody space a metallic implant selected from rods and pedicle screws or plates and pedicle screws by attachment thereof to adjacent vertebrae; injecting into the anterior portion of the interbody space an effective amount of a calcium phosphate-based bone substitute; and allowing the bone substitute to set in vivo.

The invention methods for performing spinal fusions can be performed by using either an anterior, posterior, or posterolateral approach to the interbody space. The posterolateral approach (unilateral or bilateral) reduces surgical morbidity over an anterior approach, but caution is required while working around the cauda equina and exiting nerve roots in the spinal canal. Posterior access and visualization of the interbody space is more limited than with the anterior approach, but many spinal surgeons are trained in how to deal with those circumstances.

The anterior approach for the anterior portion of the 360° fusion can be done with an open retro-peritoneal technique, or endoscopically. Although approaching the spine anteriorly can lead to a higher risk of complications and more blood loss, visualization and disc access is greatly improved over a posterior technique.

The surgical site(s) can be closed using standard suturing techniques.

A “suitable interbody space” as the term is used in the application and claims herein means the space between adjacent vertebrae where a disc resides in a healthy spine but which is either at least partially devoid of disc material due to wear and tear on the vertebral column or has been prepared using one or a combination of the above techniques, as are known in the art, to surgically create a void in the disc space.

For example, the interbody space can be prepared, as needed, by combining a nuclectomy with denuding of the caudal and cephalad vertebral end plates. Denuding the cartilaginous end-plates enables direct bone to bone substitute material contact, which is critical for bone fusion. A bilateral posterolateral approach (versus unilateral) may be needed for adequate interbody space preparation in certain posterior approach cases. In any event, preparation of the interbody space can comprise one or more techniques selected from annulatomy, nuclectomy, denuded end-plates; decorticated end-plates; and intradiscal electrothermal treatment.

For preparation of the posterior portion of the interbody space, the posterolateral gutter is decorticated and covered by bone and/or a bone substitute. Posterior instrumentation can then by applied to the spine utilizing plates or rods secured by pedicle screws to adjacent vertebral bodies.

A “subject” as the term is used herein is any mammal, including zoo, farm and domestic animals and humans.

An “effective amount” of the injectable calcium-phosphate-based bone substitute as the term is used herein is an amount effective to accomplish fusion of vertebrae adjacent to the interbody site in the subject.

“Setting time” as the term is used herein is the time after which a 1 mm diameter pin with a load of ¼ pound can be inserted only 1 mm deep into the surface of a CPC paste, as determined using ISO 1566, a method commonly used for measuring the setting time of dental zinc phosphate cements as well as CPC.

“Working time” as the term is used herein means the time after which a CPC paste becomes too viscous to be stirred. Generally working time is a few minutes shorter than setting time.

After setting for about 30 minutes, the bone substitute suitable for use in the invention methods has a minimum compressive strength of about 10 MPa and a minimum compressive strength of 25 MPa is obtained within 24 hours after injection or after exposure to physiological conditions. The compressive strength herein is as determined using ASTM F451-99, a method that is commonly used for the compressive strength measurement of CPC.

The injectable calcium phosphate-based bone substitute is introduced into the anterior portion of the interbody space in the invention methods using a syringe, catheter, cannula, or the like. An injectable calcium phosphate-based bone substitute suitable for use in the invention methods will have viscosity capable of flowing through a 24 gauge needle, or larger, and working and setting times of about 5 to about 30 minutes. After setting for about 30 minutes, the suitable bone substitute has a minimum compressive strength of about 10 MPa, or a minimum of 25 MPa compressive strength within 24 hours after injection. Additionally, when solidified, the bone substitute can have a porosity of about 20% to about 50% by volume as measured using ASTM C830-00 water saturation technique.

The injectable calcium phosphate-based bone substitute having these characteristics can consist essentially of calcium phosphate, for example being a substantially monolithic tetracalcium phosphate (Ca₄(PO₄)₂O). The calcium phosphate may further comprise surface whiskers or fine needles of calcium phosphate, said whiskers having a length up to about 5000 nm and a width up to about 500 nm, for example, a length from about 1 nm to about 2000 nm and a width from about 1 nm to about 200 nm. Alternatively, the suitable calcium phosphate-based bone substitute can comprise minor amounts of additional substances, such as Na₃PO₄; Na₂HPO₄; NaH₂PO₄; Na₄HPO₄.7H₂O; Na₃PO₄.12H₂O; H₃PO₄; CaSO₄; (NH₄)₃PO₄; (NH₄)₂HPO₄; (NH₄)H₂PO₄; (NH₄)₃PO₄.3H₂O; NaHCO₃; CaCO₃; Na₂CO₃; KH₂PO₄; K₂HPO₄; K₃PO₄; CaF₂: SrF₂; Na₂SiF₆; Na₂PO₃F, and the like. The suitable bone substitute can also comprise an amount of one or more active agents suitable to promote bone growth, such as a growth factor, a bone morphology protein, or a pharmaceutical carrier therefor.

Examples of suitable calcium phosphates that can be used in preparation of the injectable calcium phosphate-based bone substitutes used in the invention methods include, but are not limited to, Ca₄(PO₄)₂O, CaHPO₄. 2H₂O, CaHPO₄, Ca₈H₂(PO₄)₆. 5H₂O, alpha-Ca₃(PO₄)₂, beta-Ca₃(PO₄)₂, Ca₂P₂O₇, Ca₂H₂P₂O₈, and the like.

Calcium-phosphate-based cements and bone substitutes suitable for use in the invention methods, and methods for their preparation, are described, for example in U.S. Pat. Nos. 6,379,453 B1 and 6,616,742 and in co-pending U.S. patent application Ser. No. 09/351,912, filed Jul. 14, 1999; Ser. No. 09/941,576, filed Aug. 30, 2001; Ser. No. 10/179,879, filed Jun. 26, 2002; and Ser. No. 10/328,019, filed Dec. 26, 2002, each of which is incorporated herein by reference in its entirety.

Although the invention has been described with respect to specific embodiments, it will be understood that modifications and variations are encompassed within the spirit and scope of the invention. Accordingly, the invention is limited only by the following claims.

1. A method for performing one or more spinal fusions in a subject comprising introducing an effective amount of an injectable calcium phosphate-based bone substitute into one or more interbody spaces in the subject by injection through a syringe, catheter, or cannula to facilitate single, or multi level spinal fusion.

2. The method of claim 1, wherein the spinal fusion is in a segment of the spine selected from cervical, thoracic, lumbar, lumbosacral and SI joint, and combinations thereof.

3. The method of claim 1, wherein the bone substitute is injected into the one or more interbody spaces by an approach selected from posterior, posterolateral, anterior, anterolateral and lateral approaches, and combinations thereof.

4. The method of claim 1, wherein the bone substitute transforms from a viscous consistency to a solid consistency over time.

5. The method of claim 1, wherein the method facilitates the anterior fusion of vertebrae without the use of pre-formed interbody spacers, cages, dowels or plugs consisting of a biologic or non-biologic material.

6. An the method of claim 1, wherein the method does not include posterior spine fixation.

7. The method of claim 1, wherein the injectable bone substitute is bioresorbable, allowing ingrowth of autologous bone during resorption.

8. The method of claim 1, wherein the injectable bone substitute sets in the interbody space and is maintained in the body as a solid for an extended period of time.

9. The method of claim 8, wherein the extended period of time is up to and including the duration of the life of the subject.

10. The method of claim 1, wherein the calcium phosphate in the bone substitute consists essentially of substantially monolithic tetracalcium phosphate (Ca₄(PO₄)₂O).

11. A method for performing one or more spinal fusions on a subject comprising: placing in the posterior portion of at least one suitable interbody space a metallic implant selected from rods and pedicle screws or plates and pedicle screws by attachment thereof to adjacent vertebrae;
injecting into the anterior portion of the interbody space an effective amount of a calcium phosphate-based bone substitute; and
allowing the bone substitute to solidify in vivo.

12. The method of claim 11, wherein the spinal fusion is in a segment of the spine selected from cervical, thoracic, lumbar, lumbosacral and SI joint, and combinations thereof.

13. The method of claim 11, wherein the bone substitute is injected into the one or more interbody spaces by an approach selected from posterior, posterolateral, anterior, anterolateral and lateral approaches, and combinations thereof.

14. The method of claim 11, wherein the bone substitute transforms from a viscous consistency to a solid consistency over time.

15. The method of claim 11, wherein the method facilitates the anterior fusion of vertebrae without the use of pre-formed interbody spacers, cages, dowels or plugs consisting of a biologic or non-biologic material.

16. The method of claim 11, wherein the method does not include posterior spine fixation.

17. The method of claim 11, wherein the injectable bone substitute is bioresorbable, allowing ingrowth of autologous bone during resorption.

18. The method of claim 11, wherein the injectable bone substitute solidifies in the interbody space and is maintained in the body as a solid for an extended period of time.

19. The method of claim 18, wherein the extended period of time is up to and including the duration of the life of the subject.

20. The method of claim 11, wherein the injectable bone substitute develops a minimum compressive strength of 10 MPa after setting for about 30 minutes after injection.

21. The method of claim 20, wherein a minimum compressive strength of 25 MPa develops in the bone substitute within 24 hours after injection.

22. The method of claim 11 wherein the bone substitute has a setting time of about 30 minutes.

23. The method of claim 11 wherein the bone substitute has a porosity of about 20% to 50% by volume upon solidifying in vivo.

24. The method of claim 11 wherein the calcium phosphate in the bone substitute consists essentially of substantially monolithic tetracalcium phosphate (Ca₄(PO₄)₂O).
